(19) United States
(12) Patent Application Publication
(10) Pub. No.: US 2008/0058846 A1
(43) Pub. Date: Mar. 6, 2008

(54) Mechanical tissue morcellator

(76) Inventor: Khashayar Vosough, Franklin Lakes, NJ (US)

Correspondence Address: FOX ROTHSCHILD LLP, 100 PARK AVENUE, SUITE 1500, NEW YORK, NY, 10017, US

(21) Appl. No.: 11/513,727

(22) Filed: Aug. 31, 2006

(51) Int. Cl. A61B 17/32

(52) U.S. Cl. 606/180

(57) ABSTRACT

An improvement to a tissue removal device, that is, the addition of an adjustable protective guard and/or a retractable protective guard on the blade guard sheath of a mechanical tissue morcellator is presented. The retractable protective guard can be fixedly located at the distal end of a blade guard sheath and can be openly operated by a spring loaded means or a trigger means. The adjustable protective guard is moveably mounted in close contact around the blade sheath and may be positioned at any point along the length of the blade guard sheath in either direction. The addition of the adjustable protective guard and the retractable protective guard on the blade guard sheath of the mechanical tissue morcellator act to control and direct positioning of the tissue morcellator within the body cavity of the patient.

### FIELD OF THE INVENTION

The present invention is generally directed to an improvement of a tissue removal device, and more particularly, to the addition of adjustable and/or retractable protective guards on the blade guard of a mechanical tissue morcellator.

### DESCRIPTION OF THE RELATED ART

The process of minimally invasive surgery has been augmented by specialized tools for decades. In recent years, however, special medical equipment and/or electronic tools have been developed to aid surgeons. For example, special surgical instruments handled via tubes inserted into the body through small openings, fiber optic cables, and miniature video cameras to name a few. This equipment has allowed for features and advantages such as: visual magnification—use of a large viewing screen improves visibility; stabilization—electromechanical damping of vibrations, due to machinery or shaky human hands; and reduced number of incisions needed to perform the surgery.

In modern surgery access to the organ is provided by inserting one or more trocar and cannula to the tissue cite. The images of the interior of the body are transmitted to an external video monitor and the surgeon has the possibility of making a diagnosis, visually identifying internal features and acting surgically on them. If need be, one or more laparoscopes can be used to view the tissue to be removed, and additional port sites can also be used to gain access to the tissue to sever it from tissue which is to remain.

A laparoscope consists of a Hopkins rod lens system, that is usually connected to a videocamera—single chip or three chip, a fiber optic cable system connected to a ‘cold’ light source, halogen or xenon, to illuminate the operative field, inserted through a 5 mm or 10 mm cannula to view the operative field. Additional 5-10 mm thin instruments can be introduced by the surgeon through side ports.

Laparoscopic surgery, also called keyhole surgery (when natural body openings are not used), band-aid surgery, or minimally invasive surgery (MIS), is a surgical technique. With the advent of laparoscopic and endoscopic surgery, surgical patients are benefiting from shorter hospitalization, less pain and scarring, and generally better outcomes.

Nonetheless, even with such surgical technique, the entry incision must still be sized to allow removal of the severed tissue and, therefore, the reduction in entry incision size is rather limited even in more modern or recently developed surgical procedures.

One system which has been developed to overcome this limitation is described in Laparoscopic Nephrectomy: A review of 16 Cases, Surgical Laparoscopy & Endoscopy, Vol. 2, No. 1, pp. 29-34 (Raven Press, Ltd., 1992), the disclosure of which is expressly incorporated herein in its entirety. This publication describes a method for removing renal tissue using a mechanical morcellator. In this method of removal, the kidney and associated renal tissue, after being severed from the ureter, renal arteries, and veins, are placed in an impermeable containment bag. The neck of the bag is closed, withdrawn from a laparoscopic port site, and reopened to gain access to the tissue contained therein. Thereafter, with the tissue remaining in the bag within the patient's body, a morcellator cutting head is introduced into the bag and activated, fragmenting and aspirating the renal tissue. The aspirated tissue is retained within a filtering chamber within the morcellator handle, which must be cleaned following the surgical procedure.

Although the morcellation device and method disclosed in this reference represents an improvement over tissue removal methods which require a large entry incision, there is a mechanical morcellator described in U.S. Pat. Serial No. 5,520,634, the disclosure of which is also expressly incorporated herein in its entirety. The mechanical morcellator described therein provides a relatively movable cutting head and includes means to prevent the unintentional actuation of the morcellator cutting head.

However, the problem with the mechanical morcellator described in this U.S. patent and other non-patented mechanical morcellators currently on the market is that there is the possibility of causing severe harm or injury to the patient if the blade of the morcellator moves beyond the tissue which is to be removed. That is, healthy tissue could be injured or destroyed resulting in serious harm or injury to a patient should the morcellator move beyond the tissue to be removed. Accordingly, there is a need to ensure that the positioning of the blade of the mechanical morcellator is proper and steady, and not subject to inadvertent movement.

### SUMMARY OF THE INVENTION

In accordance with the present invention there is provided an improved mechanical morcellator, which is operable to fragment and aspirate tissue, having a moveable and/or retractable protective guards to allow proper positioning and prevent unintended movement. In addition, the mechanical morcellator of the present invention provides the various functional and structural features in an ergonomically designed “pistol grip” handle which facilitates the surgeon's operation and manipulation of the morcellator.

The mechanical morcellator includes a rotary cutting blade, which communicates with suction via a hollow drive tube, and a cutting head extension means. The cutting blade is driven by a variable speed electric motor via the drive cable, the speed of the motor being preset by a user-manipulated control box. The cutting blade extension means includes a blade guard sheath which extends between the morcellator body and the cutting blade. The sheath covers the cutting blade when in a first position and reveals the cutting blade when in a second position. Means are provided to allow user manipulation or adjustment of the sheath position relative to the cutting blade.

The blade guard sheath has attached to it a circular adjustable protective guard which completely encircles the blade guard sheath. The adjustable protective guard can be positioned by the surgeon closer to or further away from either the morcellator body or the cutting blade as the case may be desired. In addition, the blade guard sheath may have a series of retractable protective guards towards the tip of the blade guard sheath closest to the cutting blade. The retractable protective guards may be spring loaded or activated by a trigger means.

In further accordance with the present invention, means are provided to prevent the unintentional or accidental actuation of the cutting head. The mechanical morcellator also includes suction control means. The suction control means includes a valve which is integral with the trigger and coordinated with the cutting head operation to limit suction flow to the cutting head when the cutting head is inoperable, while permitting full suction flow thereto when the head is operating.

The present invention, including its features and advantages, will become more apparent from the following detailed description with reference to the accompanying drawings.

### BRIEF DESCRIPTION OF THE DRAWINGS

FIG. 1 illustrates, in cross section, a mechanical tissue morcellator according to the prior art.

FIG. 2 illustrates an adjustable guard placed on the blade guard of the mechanical tissue morcellator in which the cutting blade is extended, according to an embodiment of the present invention.

FIG. 3 illustrates a retractable fixed guard in a closed position placed on the blade guard of the mechanical tissue morcellator in which the cutting blade is retracted, according to an embodiment of the present invention.

FIG. 4 illustrates a retractable fixed guard in an open position placed on the blade guard of the mechanical tissue morcellator in which the cutting blade is extended, according to an embodiment of the present invention.

### DETAILED DESCRIPTION OF THE PREFERRED EMBODIMENT

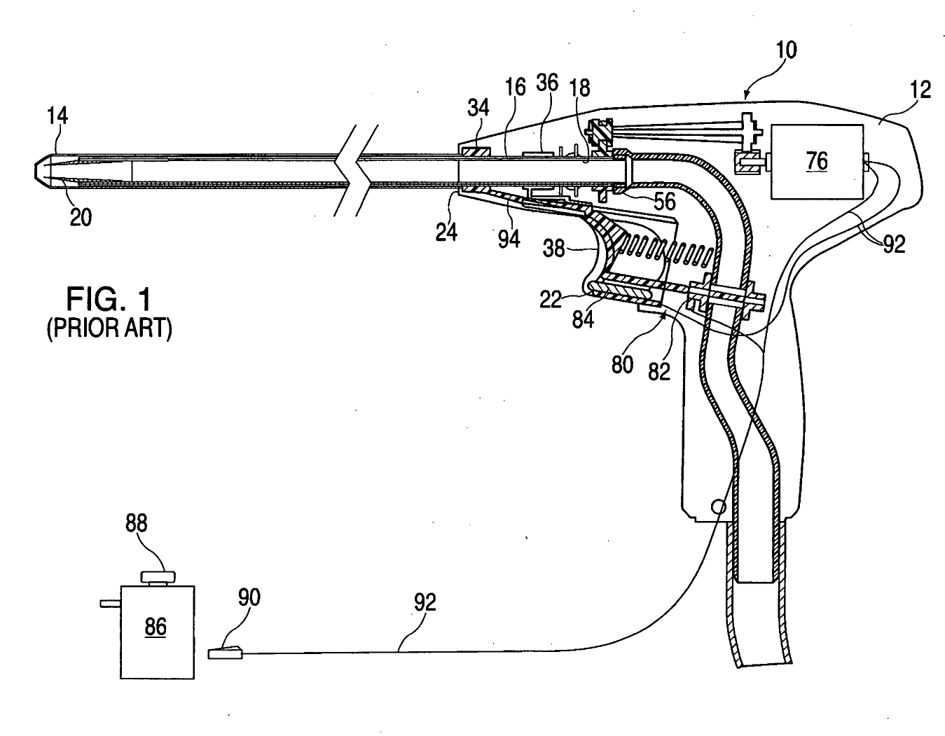

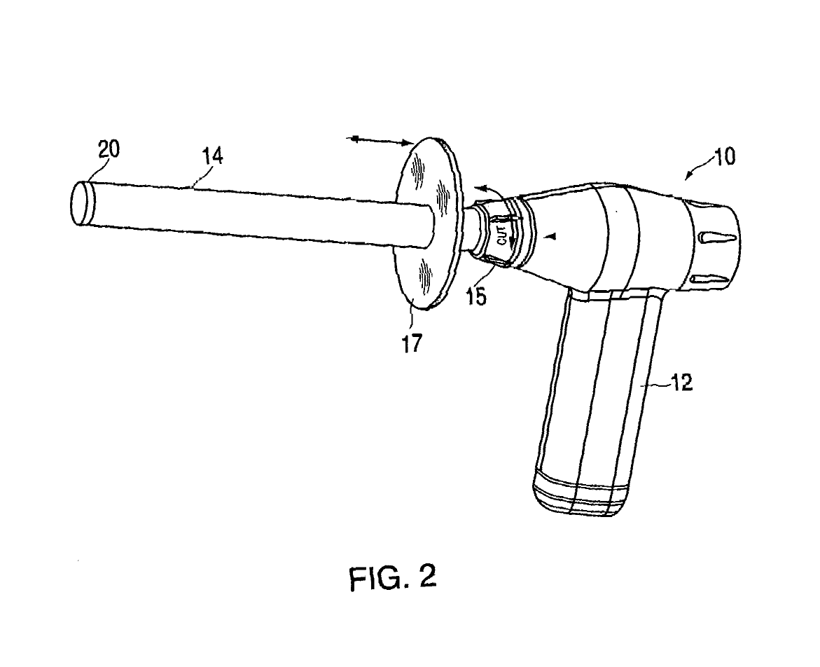

FIG. 1 of the prior art and FIGS. 2 through 4 according to the present invention illustrate differing embodiments of a mechanical tissue morcellator 10 to generally include a handle 12, a blade sheath 14, a drive tube 18, and a rotary cutting blade 20. Additionally, a barrel 16 and a trigger 22 may generally be included.

The handle 12, which is preferably formed in two mating halves of polycarbonate or ABS plastic, provides a front aperture 24 through which the blade sheath 14, barrel 16, and drive tube 18 extend. The blade sheath 14 is rigidly mounted to the handle to prevent the sheath from rotating with the drive tube 18 and/or the barrel 16. In one embodiment of the construction, the barrel 16 surrounds the drive tube 18 and extends outwardly therewith from the handle 12 to the cutting blade 20. In an alternative embodiment, the drive tube 18 surrounds the barrel 16 and extends outwardly therewith from the handle to the cutting blade 20.

At the front aperture 24, according to different embodiments, either the drive tube 18 is rotatably received within the barrel 16, or the barrel 16 is rotatably received within the drive tube 18. Both of which are then slidably received within the sheath 14. As illustrated, the sheath 14, barrel 16, and drive tube 18 are coaxial and generally tubular in shape.

Preferably, the barrel 16 is stainless steel, having an outside diameter of about 0.375 inches and a wall thickness of about 0.016 inches, the drive tube 18 is plastic, having an inside diameter of about 0.300 inches and an outside diameter of about 0.330 inches, and the sheath 14 is TEFLON or ethylene having an outside diameter of about 0.410 inches and a wall thickness of about 0.010 inches. There is a gap of about 0.045 inches between the barrel 16 and the drive tube 18, allowing the drive tube to rotate without frictional interference with the barrel.

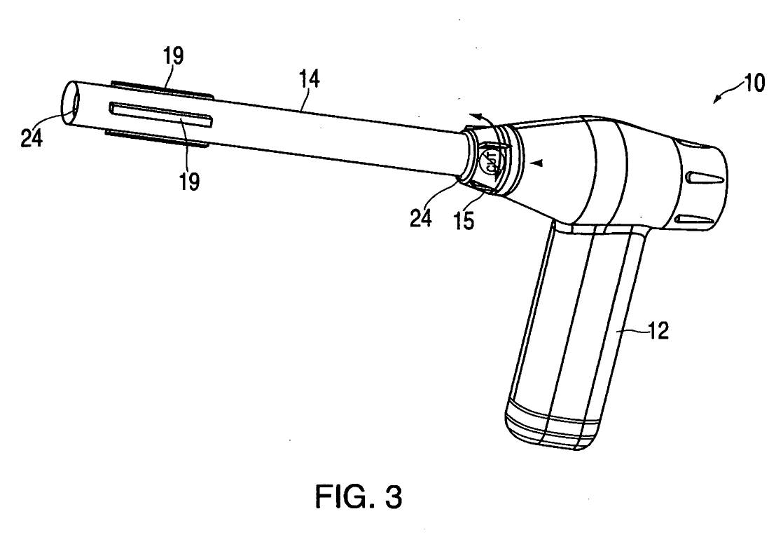

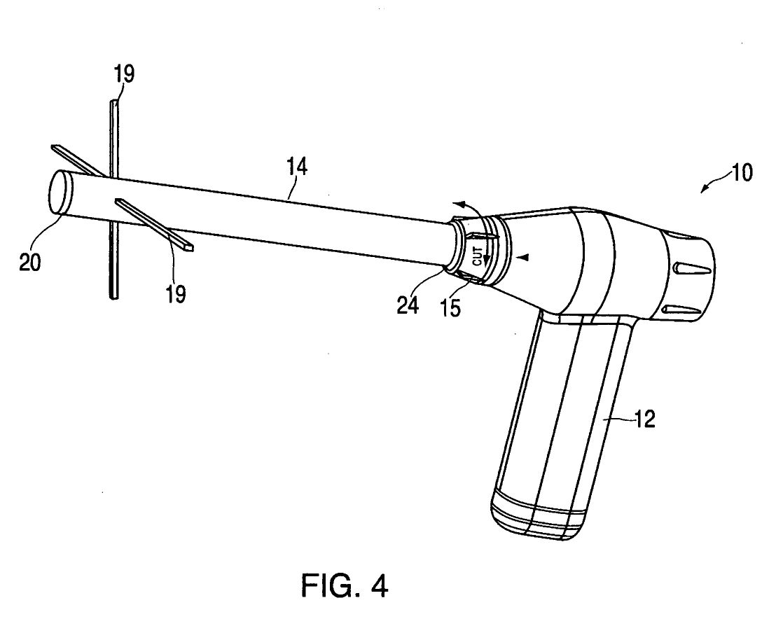

The barrel 16 is surrounded by the sheath 14 which extends outwardly therewith towards the cutting blade 20. The sheath 14 has a proximal end and a distal end. At the proximal end is switch 15 which is rotateably positional in a “cut” and “no cut” position. Movement between the two switch positions causes the cutting blade 20 and barrel 16 to extend or retract, respectively, from the sheath 14. Accordingly, the distal end of the sheath 14 generally surrounds the cutting blade 20 while the barrel 16 is in the inward or “no cut” position (see FIG. 3). Likewise, when the barrel 16 is in the outward or “cut” position, the cutting blade 20 is extended and exposed (see FIGS. 2 and 4).

The cutting blade 20 can either be frusto-concial in shape, as shown in FIG. 1, or cylindrical (tubular) in shape, as shown in FIGS. 2-4. Additionally, dependent upon the embodiment, the cutting blade 20 can be attached or mounted to either the drive tube 18 or the barrel 16. Preferably, the cutting blade has an outside diameter of about 0.300 inches to generally match the inside of the drive tube 18 when mounted thereby, or approximately 0.400 inches to match the outside of the barrel 16 when mounted thereby.

Referring specifically now to FIG. 2, an adjustable guard 17 is positioned around the blade sheath 14. Adjustable guard 17 is in close contact with the sheath 14, but is mounted such that it is slideably moveable along the length of the sheath 14. In this manner then, a surgeon can adjust the positioning of the adjustable guard 17 moving it between the distal and the proximal end, or any place in between. Such moveability of the adjustable guard along the extremity of the sheath 14 allows for setting of a maximum allowable length of the sheath 14 to be inserted into the abdominal body cavity of the patient. The adjustable guard 17 can be of any size and shape. Preferably, the adjustable guard is circular in shape and has a radius of the approximately 1 inch with a thickness of an ⅛ of an inch. In addition, it is to be understood that the slideablility of the adjustable guard allows for removable of the adjustable guard 17 from the distal end of the sheath 14. In addition, such removability of the adjustable guard allows for it to be sterilized and reused on different morecellators.

Referring specifically now to FIGS. 3 and 4, a retractable guard 19 is shown in both its retracted and extended positions. As shown in the Figures, preferably the retractable guard 19 is fixedly positioned towards the distal end of the sheath 14. Alternatively, however, it is possible to allow for the exact positioning of the retractable guards to be set by the surgeon to create a safety distance determinative of which the cutting blade 20 can extend into the inner tissues of the patient. In other words, upon positioning by the surgeon the retractable guards 19 at the distal end of the sheath 14 act to prevent the blade 20 from cutting too far into tissue as the retractable guard 19 will prevent the sheath 14 and blade 20 from moving beyond the preset distance from the distal end.

In alternative embodiments of the invention, the retractable guards 19 may be of different shapes, and indeed may be sized and shaped to fit particular areas of the body to be operated on. For instance, the retractable guards 19 may, when opened, be shaped and operate much like a hand-held fan wherein the guards interlock with one another. Alternatively, the guards may be constructed such that they are narrower or wider at either top or bottom as required by the surgical situation.

The opening of the retractable guards 19 may be activated by either a spring loaded means or a trigger means. In the instance where opening of the retractable guards is a spring loaded means, the springs (not shown) are positioned such that the retractable guards 19 are biased in a open position, as shown in FIG. 4. Pressure on the retractable guards 19 towards the distal end of the sheath 14 will cause the guards to retract. Thus during a surgical insertion, the surgeon may manually depress the retractable guards towards the distal end of the sheath 14 for insertion of the morcellator 10 into the entry point of the patient's body. As the distal end of the sheath 14 passes into a cavity in the interior of the patient's body, the spring loaded means causes the retractable guards to open. Upon exiting the insertion point of the patient's body, pressure by the surrounding tissue wall overcomes the bias of the spring loaded means and causes the retractable guards 19 to fold back to their retracted state.

In an alternative embodiment, the opening of the retractable guard 19 may be activated by a trigger means. In this case, a catch or hook (not shown) can be set to hold or clasp the distal end of the protective guard 19 itself to bias against the spring bias. Thus, the catch or hook operates to bias the protective guard 19 in a closed, or retracted, position as shown in FIG. 3. The catch or hook can be released upon activation of the trigger.

When the trigger 22 is in an at-rest or initial position (see FIG. 1), the switch 80 (i.e. the first and second contacts 82 and 84) represent an open circuit. Thus, no current is supplied to the motor 76, and the drive tube 18 and cutting head 20 are stationary. The motor 76 is electrically connected to power via the switch 80 and a transformer or control box 86 when the trigger 22 is in the inward-most or full-retracted position, as will be described more fully hereafter.

The control box 86 includes a rotary pot 88 to allow a user to preset the maximum power available to the morcellator 10 and, hence, the maximum speed of the cutting head 20. Electrical connectors 90 and appropriate lengths of electrical conductors 92 are between the control box 86 and the switch 80 and motor 76. Preferably, the conductors 90 are four-wire cables and the connectors 92 are four-pin connectors.

The trigger 22 nests within a track provided by the handle 12, and is manually movable by the surgeon, total range of travel being generally equal to the sheath travel (i.e. about 0.435 inches). The trigger 22, which is preferably formed out of plastic, includes the metal trigger latch 38 which slidably extends out of a slot in the trigger 22. The trigger 22 also includes an outwardly extending portion 94 which terminates in the cylindrical sheath receptacle 34.

The outwardly extending portion 94 defines an opening through which a hooked terminal end 96 of the trigger latch 38 extends. The hooked terminal end of the trigger latch 38 is operable to releasably engage the first and second latching surfaces.

The trigger latch 38, which is resiliently biased to extend through the trigger 22, pivots about a pivot point. The second contact 84, which is slidably received by the trigger 22, is press fit into the trigger latch 38 and moves therewith. When the trigger 22 and trigger latch 38 are in the at-rest position shown in FIG. 1, the hooked terminal end is in engagement with the first latching surface, there is a gap of about 0.075 inches between the trigger latch 38 and the trigger 22, and the first and second contacts 82 and 84 are separated by approximately 0.496 inches. When the trigger 22 and trigger latch 38 are in the inwardmost or rearwardmost position, the trigger and trigger latch are generally in contact, and the first and second contacts 82 and 84 are in engagement.

As can be seen from the above disclosure, the addition of the adjustable protective guard allows a surgeon to set a limit on the distance of how far the distal end of the blade guard sheath of the mechanical tissue morcellator will penetrate through the entry point into a patient's body cavity. This allows for additional pre-surgery command and control of the extent of internal body cavity entry. In addition, the adjustability feature of the adjustable protective guard allows for the surgeon to make in-surgery corrections as to such internal entry distances.

As can also be seen from the above disclosure, the addition of the retractable protective guards on the blade guard sheath of the mechanical tissue morcellator protects against injury to the patient by accidental slippage in the positioning of the morcellator within the body cavity of the patient. That is, the positioning of the retractable protective guards in their open position confirm and direct the extent of the distance that the blade will be allowed to cut into the tissue at the point of cutting within the body cavity of the patient. Accordingly, the addition of the adjustable protective guard and/or the retractable protective guard on the blade guard sheath of the mechanical tissue morcellator allows for additional safety features that otherwise are not present in the patented and unpatented tissue morecellators currently in use.

In the foregoing description, the method and apparatus of the present invention have been described with reference to a specific example. It is to be understood and expected that variations in the principles of the method and apparatus herein disclosed may be made by one skilled in the art and it is intended that such modifications, changes, and substitutions are to be included within the scope of the present invention as set forth in the appended claims (if any are included). The specification and the drawings are accordingly to be regarded in an illustrative rather than in a restrictive sense.

What is claimed is:

1. A mechanical tissue morcellator having a blade sheath for removing tissue from a patient's body, wherein the improvement comprises:
an adjustable protective guard slidably movable along the length of the blade sheath; and
at least one retractable protective guard hinged at a distal end of the blade sheath,
wherein the adjustable protective guard prevents insertion of the morcellator beyond the point the adjustable protective guard is positioned along the blade sheath, and
further wherein the at least one retractable protective guard prevents movement of the morcellator further into cut tissue beyond the point the retractable protective guard is positioned along the blade sheath.

2. The improved mechanical tissue morcellator according to claim 1, wherein the at least one retractable protective guard is biased towards an open position.

3. The improved mechanical tissue morcellator according to claim 1, wherein once the at least one retractable protective guard is in an open position, the at least one retractable protective guard can be closed only by withdrawing the morcellator from the patient's body.

4. The improved mechanical tissue morcellator according to claim 1, wherein the at least one retractable protective guard is fixed to the blade sheath at the hinge point.

5. The improved mechanical tissue morcellator according to claim 1, wherein the at least one retractable protective guard is positionable along the length of the blade sheath.

6. The improved mechanical tissue morcellator according to claim 1, wherein the at least one retractable protective guard is operationally openable by one of a means of a spring bias and a means of a trigger.

7. The improved mechanical tissue morcellator according to claim 1, wherein the adjustable protective guard can be removed from the blade sheath.

8. An apparatus for removing tissue or foreign objects from a patient's body, comprising:
a cutting blade for cutting tissue or foreign objects;
a blade sheath in which the cutting blade may be retracted;
an adjustable protective guard slidably movable along the length of the blade sheath; and
at least one retractable protective guard hinged at a distal end of the blade sheath,
wherein the adjustable protective guard prevents insertion of the cutting blade beyond the point the adjustable protective guard is positioned along the blade sheath, and
further wherein the at least one retractable protective guard prevents movement of the cutting blade further into cut tissue beyond the point the retractable protective guard is positioned along the blade sheath.

9. The apparatus according to claim 8, wherein the at least one retractable protective guard is biased towards an open position.

10. The apparatus according to claim 8, wherein once the at least one retractable protective guard is in an open position, the at least one retractable protective guard can be closed only by withdrawing the apparatus from the patient's body.

11. The apparatus according to claim 8, wherein the at least one retractable protective guard is fixed to the blade sheath at the hinge point.

12. The apparatus according to claim 8, wherein the at least one retractable protective guard is positionable along the length of the blade sheath.

13. The apparatus according to claim 8, wherein the at least one retractable protective guard is operationally openable by one of a means of a spring bias and a means of a trigger.

14. The apparatus according to claim 8, wherein the adjustable protective guard can be removed from the blade sheath.
